(19) United States
(12) Patent Application Publication
(10) Pub. No.: US 2022/0017846 A1
(43) Pub. Date: Jan. 20, 2022

(54) DEVICE FOR ASSESSING MECHANICAL STRAIN INDUCED IN OR BY CELLS

(71) Applicant: Minetas B.V., Leiden (NL)

(72) Inventors: Paul VULTO, Leiden (NL); Sebastiaan Johanes TRIETSCH, Leiden (NL); Todd Peter BURTON, Leiden (NL)

(73) Assignee: Mimetas B.V., Leiden (NL)

(21) Appl. No.: 17/298,314

(22) PCT Filed: Nov. 27, 2019
(86) PCT No.: PCT/EP2019/082803

§ 371 Date: May 28, 2021

(30) Foreign Application Priority Data
Nov. 28, 2018 (NL) 2022085

(51) Int. Cl. C12M 3/06; C12M 1/42; C12M 1/34; C12M 1/32; C12M 1/00; C12M 1/12; C12M 1/02

(52) U.S. Cl. CPC C12M 23/16; C12M 35/04; C12M 41/40; C12M 23/12; C12M 27/00; C12M 23/26; C12M 23/34; C12M 23/38; C12M 25/14; C12M 23/22

(57) ABSTRACT

A microfluidic device comprising a microfluidic network is described. The device comprises a base, a microfluidic channel and a cover, and the base comprises a diaphragm forming at least part of an inner surface of the microfluidic channel. The device finds use in methods for assessing mechanical strain induced in or by cells, such methods also being described.

### FIELD OF THE INVENTION

The present invention relates to a microfluidic device, and to methods of inducing or assessing mechanical strain in cells using the microfluidic device.

### BACKGROUND OF THE INVENTION

In a drive towards attempting to simulate ever more physiologically relevant conditions in cell culture, numerous models have been developed to simulate, for example, perfusion flow, co-culture, and mechanical strain in pre-clinical cell-based models for assessing drug efficacy, ADME safety.

Microfluidics has become a popular platform technology for such in vitro cell culture models due to the inherent flow of liquids or media during use, along with advances in microengineering techniques that facilitate and enable fabrication of complex microfluidic networks. However, there is still much interest in generating models that simulate or reproduce the mechanical strain placed upon cells in, for example, the lungs or gut due to shear stresses induced by air/liquid flow from respiratory and peristaltic movements.

Solutions exist such as Emulate's Lung on a Chip, where two microfluidic channels are separated by a porous membrane, with human lung alveolar epithelial cells cultured on one side and human pulmonary microvascular endothelial cells cultured on the other side of the membrane (Science (2010) 328, p 1662-1668). As also described in WO 2010/009307 to Children's Medical Center Corporation. However, direct cell-cell contact and possible juxtacrine signalling are impeded by the presence of the membrane and the size and distribution of pores.

Alveolix (http://www.alveolix.com/technology/) has a different type of solution, as partially described by Universität Bern in WO 2015/032889. In this device, epithelial cells are cultured on a membrane that is open from the top. On the bottom side, the membrane connects to a microfluidic channel that has a diaphragm underneath that applies stretch to the first membrane upon actuation. Also in this example, cells are grown upon an artificial surface.

To date no solution exists that is directed to the culturing of different cell types in a setup that simultaneously allows juxtacrine interaction between e.g. a microvascular network and an epithelial cell layer, while still being in a position to apply stretch to the epithelial cell layer. This requires a totally different solution that is devoid of artificial membranes.

There exists a need for improved devices for simulating mechanical strain in cells.

### SUMMARY OF THE INVENTION

In a first aspect of the invention there is provided a microfluidic device, comprising:
a microfluidic network, the microfluidic network comprising:
a base, a microfluidic channel, and a cover;
wherein the base comprises a non-porous diaphragm forming at least part of an inner surface of the microfluidic channel and wherein the microfluidic channel comprises a sub-volume defined at least in part by the diaphragm and by a capillary pressure barrier in the microfluidic channel.

In a second aspect of the invention there is provided a method to assess mechanical strain induced by cells, comprising:
introducing one or more types of cells or cell aggregates into the microfluidic network of a microfluidic device according to the first aspect;
optionally culturing the one or more types of cells or cell aggregates; and
monitoring deflection of the diaphragm using one or more electrodes, sensors, probes, reference markers for monitoring diaphragm movement, ferromagnetic particles, or antibodies disposed on or operatively connected to the diaphragm.

In a third aspect of the invention there is provided a method of subjecting one or more types of cells or cell aggregates to mechanical strain i.e. inducing mechanical strain in the one or more types of cells or cell aggregates, comprising:
introducing one or more types of cells or cell aggregates into the microfluidic network of a microfluidic device according to the first aspect;
optionally culturing the one or more types of cells or cell aggregates; and
subjecting the one or more types of cells or cell aggregates to mechanical strain by applying a positive pressure or a negative pressure to the diaphragm.

According to a fourth aspect of the present invention, there is provided an assay plate, comprising the device of the first aspect provided with a gel confined by the capillary pressure barrier to the sub-volume of the microfluidic channel, optionally wherein the gel comprises one or more cells or cell aggregates.

According to a fifth aspect of the present invention, there is provided a kit, comprising:
the assay plate of the third aspect of the invention; and
one or more pro-angiogenic compounds, for inducing angiogenesis.

Previous studies have demonstrated actuation of epithelia, and actuation of both epithelia and endothelia. However, the mechanical actuation of a vascularized tissue in the form of a number of microvessels interacting with the epithelium in a juxtacrine manner (i.e. direct cell-cell contact) has hitherto not been possible or demonstrated. In order to achieve this, some form of initial patterning devoid of artificial barriers such as filter membranes is required, i.e. epithelia need to be seeded in a different location with respect to endothelia, while still allowing those cells to directly interact. A device in accordance with any of the afore-mentioned aspects unexpectedly enables the mechanical actuation of a vascularized tissue, thus opening up the development of improved in vitro or ex vivo model systems for assessing drug efficacy or ADME safety.

### Definitions

Various terms relating to the devices, methods, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

As used herein, the “a,” “an,” and “the” singular forms also include plural referents unless the content clearly dictates otherwise. Thus, for example, reference to “a cell” includes a combination of two or more cells, and the like.

As used herein, “about” and “approximately”: these terms, when referring to a measurable value such as an amount, a temporal duration, and the like, are meant to encompass variations of ±20% or ±10%, more preferably ±5%, even more preferably ±1%, and still more preferably ±0.1% from the specified value, as such variations are appropriate to perform the disclosed methods.

As used herein, “comprising” is construed as being inclusive and open ended, and not exclusive. Specifically, the term and variations thereof mean the specified features, steps or components are included. These terms are not to be interpreted to exclude the presence of other features, steps or components.

As used herein, “exemplary” means “serving as an example, instance, or illustration,” and should not be construed as excluding other configurations disclosed herein.

As used herein, the term “microfluidic channel” refers to a channel on or through a layer of material that is covered by a top-substrate or cover, or to a channel underneath or through a material placed onto a bottom substrate or base, with at least one of the dimensions of length, width or height being in the sub-millimeter range. It will be understood that the term encompasses channels which are linear channels, as well as channels which are branched, or have bends or corners within their path. A microfluidic channel typically comprises an inlet for administering a volume of liquid. The volume enclosed by a microfluidic channel is typically in the microliter or sub-microliter range. A microfluidic channel typically comprises a base, which may be the top surface of an underlying material, two side walls, and a ceiling, which may be the lower surface of a top substrate overlying the microfluidic channel, with any configuration of inlets, outlets and/or vents as required. The base, side walls and ceiling may each be referred to as an inner surface of the microfluidic channel, and collectively may be referred to as the inner surfaces. In some examples, the microfluidic channel may have a circular or semi-circular cross-section, which would then be considered to have one or two inner surfaces respectively.

As used herein, “diaphragm” refers to an elastomeric and/or non-porous member which is resiliently biased such that it is deformable under application of pressure, but returns to a resting state once application of pressure has ceased. References to “actuation”, “displacement”, “deflection” or “distortion” of the diaphragm are to be understood as being equivalent to “deformation” of the diaphragm.

As used herein, “droplet retention structures”, and “capillary pressure barriers” are used interchangeably, and are used in reference to features of a device that keep a liquid-air or other fluid-fluid meniscus pinned on a certain position by capillary forces. A capillary pressure barrier can be considered to divide a microfluidic channel having a volume V₀ into two sub-volumes V₁ and V₂ into which different fluids can be introduced. Put differently, a capillary pressure barrier at least partially defines a sub-volume or sub-volumes of a microfluidic channel by being located at the boundary between two sub-volumes.

As used herein, with particular reference to capillary pressure barriers, “substantially aligned with”, for example “substantially aligned with an aperture” will be understood to mean that there is no significant off-setting or displacement of the location of the capillary pressure barrier relative to a point on the perimeter of the aperture when the microfluidic device is viewed from above.

As used herein, with particular reference to capillary pressure barriers, a “closed geometric configuration” may be one in which the capillary pressure barrier is other than a linear capillary pressure barrier with two ends and instead forms a closed loop. For example, when viewed from above, a capillary pressure barrier with a closed geometric configuration may comprise a circular capillary pressure barrier, or a polygonal capillary pressure barrier, for example a triangular capillary pressure barrier, or a square capillary pressure barrier, or a pentagonal capillary pressure barrier, and so on. In some examples, a closed geometric configuration of capillary pressure barrier may also refer to two linear capillary pressure barriers arranged so as to both intersect with the same wall or walls of the microfluidic channel and thereby close off or define an area of the microfluidic channel bounded by the two linear capillary pressure barriers and the wall(s).

As used herein, the term “concentric” is to be understood as referring to any closed geometric configuration of capillary pressure barrier having a centre and not to a circular configuration or any other shape or configuration which corresponds to the shape or configuration of another capillary pressure barrier or aperture with which the capillary pressure barrier is concentric, i.e. co-centred. For example, the term “concentric” is also to be understood as referring to two linear capillary pressure barriers arranged so as to both intersect with the same wall or walls of the microfluidic channel and thereby close off or define an area of the microfluidic channel bounded by the two linear capillary pressure barriers and the wall(s) and having a centre.

As used herein, a “linear” capillary pressure barrier is not to be construed as being a straight line, but is instead to be construed as being other than a closed geometric configuration, i.e. as a line with two ends, but which may comprise one or more bends or angles. A linear capillary pressure barrier typically intersects at each end with a sidewall of the microfluidic channel.

As used herein, the terms “strain compartment” and “cell culture chamber” refer to a sub-volume of the microfluidic network, defined at least in part by a surface of a diaphragm. The sub-volume may also be defined in part by a capillary pressure barrier located in the microfluidic network.

As used herein, the term “endothelial cells” refers to cells of endothelial origin, or cells that are differentiated into a state in which they express markers identifying the cell as an endothelial cell.

As used herein, the term “epithelial cells” refers to cells of epithelial origin, or cells that are differentiated into a state in which they express markers identifying the cell as an epithelial cell.

As used herein, the term “droplet” refers to a volume of liquid that may or may not exceed the height of the microfluidic channel and does not necessarily represent a round, spherical shape. Specifically, references to a gel droplet are to a volume of gel in the strain compartment.

As used herein, the term “biological tissue” refers to a collection of identical, similar or different types of functionally interconnected cells that are to be cultured and/or assayed in the methods described herein. The cells may be a cell aggregate, and/or a particular tissue sample from a patient. For example, the term “biological tissue” encompasses organoids, tissue biopsies, tumor tissue, resected tissue material, spheroids and embryonic bodies.

As used herein, the term “cell aggregate” refers to a 3D cluster of cells in contrast with surface attached cells that typically grow in monolayers. 3D clusters of cells are typically associated with a more in-vivo like situation. In contrast, surface attached cells may be strongly influenced by the properties of the substrate and may undergo de-differentiation or undergo transition to other cell types.

As used herein, the term “lumened cellular component” refers to a biological tissue (i.e. constituted of cells) having a lumen, for example a microvessel having apical and basal surfaces.

As used herein, the term “non-porous” in connection with a diaphragm refers to a diaphragm which is substantially or completely impermeable to liquids, in particular liquids containing nutrients or waste products from cell culture experiments.

### BRIEF DESCRIPTION OF THE FIGURES

The present invention will now be described by way of example only, with reference to the Figures, in which:

FIGS. 1 to 3 show a vertical cross-section view (FIG. 1), a horizontal top view (FIG. 2), and a close up vertical cross-section view (FIG. 3) of a first possible configuration for a microfluidic network as used in a device as herein described;

FIGS. 4 to 6 show a vertical cross-section view (FIG. 4), a horizontal top view (FIG. 5), and a close up vertical cross-section view (FIG. 6) of a second possible configuration for a microfluidic network as used in a device as herein described;

FIGS. 7A to 7C show a close up vertical cross-section view of a microfluidic network as used in a device as herein described and in particular showing a diaphragm in a state of rest (FIG. 7A), in a deformed state upon negative actuation (FIG. 7B), and in a deformed state upon positive actuation (FIG. 7C);

FIGS. 8A to 8F show a schematic representation of the steps in a method as herein described;

FIGS. 9A to 9F show a schematic representation of the steps in an alternative method as herein described;

FIGS. 10A to 100 show close up vertical cross-section views of alternative configurations for a microfluidic network as used in a device as herein described;

FIGS. 11A and 11B show a gel or extracellular matrix pinned by capillary pressure barriers and aperture rims of different configurations of microfluidic networks as used in devices as herein described;

FIGS. 12 and 13 show uses of an alternative configuration of a microfluidic network as used in a device as herein described;

FIGS. 14A and 14B show alternative ways of fixing a diaphragm to the base of a microfluidic network or device as herein described;

FIG. 15 shows a plan view of a device according to the invention and consisting of a multi-well configuration of the microfluidic networks as herein described; and

FIGS. 16 and 17 show vertical cross-section views of devices as herein described and consisting of a multi-well configuration of the microfluidic networks.

### DETAILED DESCRIPTION OF THE INVENTION

### Microfluidic Device

A microfluidic device is described. The microfluidic device is preferably in a multi-array format/multi-well format to enable its use in in-vitro cell-based assays, pharmaceutical screening assays, toxicity assays, and the like; in particular in a high-throughput screening format. Such multi-well culture plates are available in 6-, 12-, 24-, 48-, 96-, 384- and 1536 sample wells arranged in a rectangular matrix, wherein in the context of the present invention a multi-array configuration of microfluidic networks as herein described are present in the microfluidic device. In one example, the microfluidic device is compatible with one or more dimensions of the standard ANSI/SLAS microtiter plate format. In an alternative embodiment the microfluidic device is in a multi-array format with dimensions of a microscope glass slide.

The microfluidic device therefore preferably has a plurality of microfluidic networks as herein described. In one example, the plurality of microfluidic networks are fluidly disconnected from each other; in other words, each microfluidic network operates independently of any other microfluidic network present on the microfluidic device.

In one example the microfluidic device comprises:
a microfluidic network, the microfluidic network comprising:
   a base, a microfluidic channel, and a cover;
      wherein the base comprises a diaphragm forming at least part of an inner surface of the microfluidic channel and wherein the microfluidic channel comprises a sub-volume defined at least in part by the diaphragm and by a capillary pressure barrier in the microfluidic channel.

In one example the microfluidic device comprises:
a microfluidic network, the microfluidic network comprising:
   a microfluidic channel comprising a cell culture chamber;
   a cover on the microfluidic channel; and
   a base on which the microfluidic channel is disposed, the base comprising:
      an aperture to the cell culture chamber and a diaphragm extending across the aperture thereby forming at least part of a floor of the cell culture chamber.

In one example the microfluidic device comprises:
a microfluidic network, the microfluidic network comprising:
   a microfluidic channel comprising a strain compartment;
   a cover on the microfluidic channel; and
   a base on which the microfluidic channel is disposed, the base comprising:
      an aperture to the cell culture chamber and a diaphragm extending across the aperture thereby forming at least part of a floor of the strain compartment.

In one example the microfluidic device comprises:
a microfluidic network, the microfluidic network comprising:
   a microfluidic channel;
   a cover on the microfluidic channel, the cover comprising an aperture to the microfluidic channel; and
   a base on which the microfluidic channel is disposed, the base comprising a diaphragm forming at least part of a floor of the microfluidic channel, wherein the diaphragm is substantially aligned with the aperture.

In one example the microfluidic device comprises:
a microfluidic network, the microfluidic network comprising:
   a microfluidic channel;
   a cover on the microfluidic channel; and
   a base on which the microfluidic channel is disposed, the base comprising a region of thinner cross-section than the surrounding portion of the base.

In one example the microfluidic device comprises:
a microfluidic network, the microfluidic network comprising:
   a base, a microfluidic channel having inner surfaces, and a cover comprising an aperture into the microfluidic channel;
wherein the microfluidic channel comprises first and second capillary pressure barriers, the first and second capillary pressure barriers each being disposed on the same inner surface and substantially aligned with and concentric with the aperture in the cover.

Generally, the microfluidic device is a microfluidic device that comprises at least a microfluidic network having a microfluidic channel. Different configurations of microfluidic channels or networks are possible within the metes and bounds of the invention, but may include for example a volume or sub-volume within or in fluid communication with the microfluidic channel, for receiving and confining a gel, for example an extracellular matrix.

The microfluidic device generally comprises a microfluidic network, each of which will now be described in detail.

### Microfluidic Network

The microfluidic network of the microfluidic device generally comprises a base, a microfluidic channel or microfluidic layer and a cover, also referred to herein as a cover layer, and can be fabricated in a variety of manners.

The base, also referred to herein as the base layer, or bottom substrate, is preferably formed from a substantially rigid material, such as glass or plastic, and serves to provide a supporting surface for the rest of the microfluidic network. In one example, the base is of the same or similar dimensions to the well area of a standard ANSI/SLAS microtitre plate. In some examples, the base comprises an aperture to the microfluidic layer or channel, across which a diaphragm as described herein extends. In some examples, the base is formed from material which is sufficiently rigid in bulk form to support the rest of the microfluidic device, but which performs as an elastomer when in the form of a thin sheet. In such examples, the base may comprise a region of thinner cross-section compared to the surrounding portions, for example the rest of the base, with that region of thinner cross-section being sufficiently elastomeric that it functions as a diaphragm as described herein.

In one example, the base layer may comprise a diaphragm sandwiched between and laminated to two sheets of etched, laser drilled or milled glass.

The base interfaces with a means to actuate the diaphragm during use of the microfluidic device. For example, the base may be configured to operatively connect the diaphragm to one or more of a source of positive or negative (air-) pressure (i.e. a pump), a physical actuator, an electromagnetic actuator; and an expandable foam. Such methods of actuating a diaphragm are known in the art and need no further discussion.

The microfluidic device or network comprises a microfluidic channel or microfluidic layer disposed on the base. In some examples, the microfluidic channel may comprise or be divided into sub-volumes, for example by the presence of a capillary pressure barrier as described herein. In some examples, the microfluidic channel may comprise a first sub-volume, which may be referred to as a strain compartment or a cell culture chamber. In some examples, the strain compartment or cell culture chamber may be defined in part by the presence of a capillary pressure barrier and/or a diaphragm in the microfluidic channel. In some examples, the diaphragm may form at least part of the surface or floor of the first sub-volume.

In some examples, the microfluidic channel further comprises a second sub-volume comprising a flow channel, and a third sub-volume that is separated from the second sub-volume by the first sub-volume. In some examples, the flow channel of the second sub-volume is an in-use flow channel. The third sub-volume may be, in-use, a second flow channel adjacent the first sub-volume, or conceptually it may be at least partly located above the first sub-volume, and only become available for filling/occupation once the first sub-volume has been filled with, for example, a gel or extracellular matrix composition. In some examples, the diaphragm forms at least part of the surface of the third sub-volume. The third sub-volume may be confined by a further capillary pressure barrier.

A typical method of fabrication of a microfluidic channel is to cast a mouldable material such as polydimethylsiloxane onto a mould, so imprinting the microfluidic channel into the silicon rubber material thereby forming a microfluidic layer. The rubber material with the channel embedded is subsequently placed on a base layer of glass or of the same material to thus create a seal. Alternatively, the channel structure could be etched in a material such as glass or silicon, followed by bonding to a top or bottom substrate (also referred to herein as a cover layer and base layer). Injection moulding or embossing of plastics followed by bonding is another manner to fabricate the microfluidic channel network. Yet another technique for fabricating the microfluidic channel network is by photo lithographically patterning the microfluidic channel network in a photopatternable polymer, such as SU-8 or various other dry film or liquid photoresists, followed by a bonding step. When referred to bonding it is meant the closure of the channel by a cover or base. Bonding techniques include anodic bonding, covalent bonding, solvent bonding, adhesive bonding, and thermal bonding amongst others.

As deduced from the various fabrication methods above, the microfluidic layer may comprise a sub-layer comprising a microfluidic channel disposed on the base layer, or is patterned in either the cover or base layer. In an in use orientation, the microfluidic sub-layer is disposed on the top surface of the base layer. The microfluidic channel may be formed as a channel through a sub-layer of material disposed on the base layer. In one example, the material of the sub-layer is a polymer placed on the base layer and into which the microfluidic channel is patterned. In some examples, the microfluidic layer comprises two or more microfluidic channels, which may be in fluidic communication with each other.

The microfluidic network comprises a cover or cover layer covering the microfluidic channel. The cover or cover layer can be formed from any suitable material as is known in the art, for example a glass layer bonded to the sub-layer comprising the microfluidic channel. In one example, the cover layer is provided with pre-formed holes or apertures at defined points. The apertures, which may be referred to herein as inlet apertures, allow for fluid communication between the microfluidic channel of the microfluidic layer and other components of the microfluidic device disposed thereon. In general the inlet apertures fulfil the function as interface with the outside world or wells disposed on top of the apertures.

The microfluidic channel may be provided with one or more additional fluid inlets, and one or more outlets or vents, as required for any particular use of the microfluidic network of the microfluidic device. In order to allow filling, emptying and perfusion of a fluid through the microfluidic network, the microfluidic channel is preferably provided with at least one inlet and at least one outlet or vent. In one example, each of the at least one inlet and at least one outlet or vent is preferably a pre-formed aperture in the cover layer. It will be understood that there typically is no geometrical distinction between an in- and outlet and that in many cases they can be used as in- or outlet interchangeably.

In some examples, the microfluidic device further comprises a top layer disposed on the above mentioned cover layer, the top layer having one, or at least one well in fluidic communication with the rest of the microfluidic device. In some examples, the top layer has a plurality of such wells, and at least one, for example at least two, for example at least three wells are in communication with a microfluidic network or channel of the device. For example, the top layer may comprise a well in fluidic communication with a microfluidic network via an inlet aperture provided in a cover layer of the microfluidic network thereby forming a SLAS compliant well plate. The well and inlet aperture may be substantially aligned with a diaphragm of a microfluidic device as described herein. In some examples, the top layer having at least one well and the microfluidic layer are integrally formed. For example, a microfluidic channel may be patterned onto the underside of an injection moulded microtiter plate having at least one well.

### Diaphragm

In some examples, the microfluidic devices of the present disclosure generally comprise a diaphragm, in the form of an elastomeric and/or non-porous membrane. These properties of the diaphragm of the present disclosure distinguish it from the types of membranes typically used for cell culture in microfluidic devices which serve as a permeable support for the cells being cultured while physically separating the cells from a perfusion channel providing nutrients and/or removing waste products, or from other cells to be co-cultured.

The function of the diaphragm of the described devices is to mimic muscle actuation in the body: for example by deflecting in a repetitive pattern to mimic breathing, peristaltic movement or heartbeat, or in a non-repetitive pattern to mimic widening or narrowing of blood vessels, or mimic muscle contraction/relaxation such as e.g. in the iris.

In some examples, the diaphragm at least partly forms an inner surface, for example a floor, of the microfluidic channel. In some examples, the diaphragm at least partly forms an inner surface, for example a floor, a sub-volume of the microfluidic channel, for example a first sub-volume and/or a second sub-volume and/or a third sub-volume. In some examples, the diaphragm at least partly forms an inner surface, for example a floor, of a cell culture chamber. In some examples, the diaphragm at least partly forms an inner surface, for example a floor, of a strain compartment. In some examples, the diaphragm is substantially aligned with an inlet aperture provided in the cover of the microfluidic device.

In some examples, the base comprises two sub-layers between which is sandwiched a sheet of elastomer. In these examples, the two sub-layers of the base have co-aligned apertures, with the elastomer extending fully across the apertures so as to form the diaphragm. In other examples, the elastomeric sheet forming the diaphragm is similarly dimensioned to the aperture and is attached to the upper surface of the base, to the lower surface of the base or to the inner side walls of the aperture using standard bonding techniques such as with an adhesive, clamping, surface tension, covalent bonding, anchoring, moulding or other manufacturing technique.

The diaphragm may be a biocompatible diaphragm, by which is meant that it is formed from an elastomer which is biocompatible and suitable for cell culture purposes. The skilled person will know what requirements are placed on a material in order for it to be considered biocompatible and suitable for cell culture, but examples may include good cytophilicity, low gas permeability, low cytotoxicity, chemical inertness, low leaching, low autofluorescence,

The diaphragm may comprise an elastomer selected from polyisoprene, polybutadiene, chloroprene, butyl rubbers, styrene-butadiene, nitrile, ethylene propylene, ethylene propylene diene, epichlorohydrin, polyacrylic rubber, silicone, polydimethylsiloxane, fluorosilicone, a fluoroelastomer, a perfluoroelastomer, a polyether block amide, chlorosulfonated polyethylene, ethylene-vinyl acetate, polyurethane, polysulfide, polyvinylidene fluoride (PVDF), ultra low density polyethylene (ULDPE), ethylene vinyl alcohol (EVOH). Examples of commercially available elastomers include Viton®, Tecnoflon®, Fluorel®, Aflas®, Dai-EI™, Tecnoflon®, Kalrez®, Chemraz®, and Perlast®. The skilled person will know that many viscoelastic materials which reversibly deform may be used as a diaphragm.

In some examples, the diaphragm is transparent or optically clear and preferably has a thickness of less than 1 mm, more preferably less than 250 μm, more preferably less than 100 μm.

In some examples, the diaphragm is a functionalised diaphragm comprising one or more electrodes, sensors, probes, reference markers for monitoring diaphragm movement, ferromagnetic particles, or adhesion molecules or antibodies for facilitating adhesion of cells to the surface of the diaphragm.

Functionalisation of the diaphragm in this way enables experiments to monitor mechanical strain emanating from cells disposed on the diaphragm, as well as controlling external actuation of the diaphragm by monitoring the extent of deformation relative to an applied actuation force.

The shape of the diaphragm and/or aperture in the base across which it extends is not limited to any particular shape but may, for example, correspond to a circle, ellipse, rectangle, rounded rectangle, dog-bone, or star.

The size of the diaphragm is typically between 1 and 2 mm for a 384 well plate layout. However, larger diaphragms might be beneficial for some applications, particularly in conjunction with for instance a 96 well microtiter plate. In this latter case, diaphragms between 2 and 4 mm or larger may be beneficial.

### Capillary Pressure Barrier

The microfluidic network of the microfluidic device may comprise a capillary pressure barrier.

In some examples, the capillary pressure barrier is substantially aligned with an aperture in the cover. In some examples, the capillary pressure barrier divides the microfluidic channel into a first sub-volume and a second sub-volume. In some examples, the capillary pressure barrier at least partially defines a sub-volume of the microfluidic channel in combination with a diaphragm.

The function and patterning of capillary pressure barriers have been previously described, for example in WO 2014/038943 A1. As will become apparent from the exemplary embodiments described hereinafter, the capillary pressure barrier, also referred to herein as a droplet retention structure, is not to be understood as a wall or a cavity which can for example be filled with a droplet comprising one or more cells or cell aggregates, but consists of or comprises a structure which ensures that such a droplet does not spread due to the surface tension. This concept is referred to as meniscus pinning. As such, stable confinement of a droplet comprising one or more cells or cell aggregates, to a sub-volume of a microfluidic channel of the device can be achieved. In one example, the capillary pressure barrier may be referred to as a confining phaseguide, which is configured to not be overflown during normal use of the cell culture device or during initial filling of a cell culture device with a first fluid. The nature of the confinement of a droplet is described later in connection with the description of the methods of the present invention.

In one example, the capillary pressure barrier comprises or consists of a rim or ridge of material protruding from an internal surface of the microfluidic channel; or a groove in an internal surface of the microfluidic channel. The sidewall of the rim or ridge may have an angle α with the top of the rim or ridge that is preferably as large as possible. In order to provide a good barrier, the angle α should be larger than 70°, typically around 90°. The same counts for the angle α between the sidewall of the ridge and the internal surface of the microfluidic channel on which the capillary pressure barrier is located. Similar requirements are placed on a capillary pressure barrier formed as a groove.

An alternative form of capillary pressure barrier is a region of material of different wettability to an internal surface of the microfluidic channel, which acts as a spreading stop due to capillary force/surface tension. In one example, the internal surfaces of the microfluidic channel comprise a hydrophilic material and the capillary pressure barrier is a region of hydrophobic, or less hydrophilic material. In one example, the internal surfaces of the microfluidic channel comprise a hydrophobic material and the capillary pressure barrier is a region of hydrophilic, or less hydrophobic material.

Thus in a particular embodiment of the present invention, the capillary pressure barrier is selected from a rim or ridge, a groove, a hole, or a hydrophobic line of material or combinations thereof. In other embodiments capillary pressure barriers can be created by a widening of the microfluidic channel or by pillars at selected intervals, the arrangement of which defines the first sub-volume or area that is to be occupied by the gel. In one example, the pillars extend the full height of the microfluidic channel.

As a result of the presence of a capillary pressure barrier, liquid is prevented from flowing beyond the capillary pressure barrier and enables the formation of stably confined volumes in the microfluidic channel, for example in one or more of the first, second or third sub-volumes, any of which may be referred to or function as a strain compartment or a cell culture chamber.

The capillary pressure barrier may be substantially aligned with an aperture in the cover layer so as to restrict spread of a droplet of fluid within the microfluidic network. In one example, the capillary pressure barrier is located on an underside of the cover layer substantially adjacent the aperture. In one example, the capillary pressure barrier is formed at least in part by the aperture itself.

In one example, the capillary pressure barrier is provided on an internal surface of the microfluidic channel facing an aperture in the cover. In a more particular embodiment the capillary pressure barrier is present on the base of the microfluidic layer or on the internal surface of the microfluidic channel substantially opposite or facing an aperture in the cover. In one example, the capillary pressure barrier is present as previously defined in order to confine a droplet of fluid to a sub-volume of the microfluidic layer aligned with an aperture of the cover.

In one example, the capillary pressure barrier defines at least in part a surface, for example a floor, of a first sub-volume of the microfluidic channel which may also be referred to as a cell culture chamber or strain compartment. The capillary pressure barrier is configured to confine a fluid to the first sub-volume of the microfluidic channel. In one example, the capillary pressure barrier comprises a closed geometric configuration. In one example, the capillary pressure barrier is concentric with the aperture of the cover layer.

In one example, the diameter or area defined by the circumference of the capillary pressure barrier is greater than the diameter or area defined by the circumference of an aperture in the cover; in other words the capillary pressure barrier is circumferential to and larger than the aperture. In another example, the diameter or area defined by the circumference of the aperture is greater than the diameter or area defined by the circumference of the capillary pressure barrier; in other words the aperture is circumferential to and larger than the capillary pressure barrier. Irrespective of the shape, in a preferred embodiment the capillary pressure barrier delineates the contact area of a droplet of liquid or gel composition comprising one or more cells or cell aggregates introduced into the microfluidic channel, i.e. being circumferential to the contact area of the droplet comprising one or more cells or cell aggregates with the base of the microfluidic channel.

In one example, the capillary pressure barrier is a substantially linear capillary pressure barrier which spans the complete width of the microfluidic channel and intersects on each end with sidewalls of the microfluidic channel.

As part of the microfluidic network, the capillary pressure barrier divides the network into at least two sub-volumes.

### Second Capillary Pressure Barrier

In some examples, the microfluidic network of the device is provided with a second capillary pressure barrier, the form and function of which is substantially as described above. For the avoidance of doubt, references to “a capillary pressure barrier” are to be understood as references to “the first capillary pressure barrier” when a second capillary pressure barrier is present in the device.

In some examples, the second capillary pressure barrier is substantially aligned with an aperture in the cover layer so as to restrict spread of a droplet of fluid within the microfluidic network. In one example, the second capillary pressure barrier is located on an underside of the cover layer substantially adjacent the aperture. In one example, the second capillary pressure barrier is formed at least in part by the aperture itself.

In one example, the second capillary pressure barrier is provided on an internal surface of the microfluidic channel facing the aperture in the cover. In a more particular embodiment the second capillary pressure barrier is present on the base of the microfluidic layer or on the internal surface of the microfluidic channel substantially opposite or facing the aperture. In one example, the second capillary pressure barrier is present as previously defined in relation to the aperture or well in order to confine a droplet of fluid to the region of the microfluidic layer aligned with the aperture.

In one example, the second capillary pressure barrier defines at least in part, in combination with the first capillary pressure barrier, a surface of the strain compartment or cell culture chamber on the base of the microfluidic layer, on the base of the microfluidic channel and/or on the diaphragm. The second capillary pressure barrier is configured, in combination with the first capillary pressure barrier, to confine a fluid to the first sub-volume comprising the strain compartment and/or cell culture chamber. In one example, the second capillary pressure barrier comprises a closed geometric configuration. In one example, the second capillary pressure barrier is concentric with the aperture of the cover layer and/or the first capillary pressure barrier. In one example, the diameter or area defined by the circumference of the second capillary pressure barrier is greater than the diameter or area defined by the circumference of the aperture and/or the first capillary pressure barrier; in other words, the second capillary pressure barrier is circumferential to and larger than the first capillary pressure barrier and/or the aperture. In one example, the second capillary pressure barrier is concentric with the first capillary pressure barrier and is within the circumference of the first capillary pressure barrier. In another example, the diameter or area defined by the circumference of the aperture is greater than the diameter or area defined by the circumference of the second capillary pressure barrier; in other words, the aperture is circumferential to and larger than the second capillary pressure barrier. Irrespective of the shape, in a preferred embodiment the second capillary pressure barrier delineates the contact area of a droplet of a liquid or gel composition comprising one or more cells or cell aggregates introduced into the strain compartment with the base of the strain compartment, i.e. being circumferential to the contact area of the droplet comprising one or more cells or cell aggregates with the base of the strain compartment.

In one example, the second capillary pressure barrier is a substantially linear capillary pressure barrier which spans the complete width of the microfluidic channel and intersects on each end with sidewalls of the microfluidic channel. In this example, the first and second capillary pressure barriers in conjunction with the walls with which they intersect may define an area which is aligned with the aperture of the cover layer, and which may also be concentric with the aperture of the cover. In this example, the first capillary pressure barrier can be considered as dividing the microfluidic network into a first sub-volume comprising the strain compartment or cell culture chamber and a second sub-volume comprising the microfluidic channel, with the second capillary pressure barrier dividing the microfluidic network into the first sub-volume comprising the cell culture chamber or strain compartment and a third sub-volume comprising a second microfluidic channel.

As part of the microfluidic network, the second capillary pressure barrier divides the network into at least two sub-volumes, the first being the first sub-volume referred to previously which comprises the strain compartment or cell culture chamber, and a third sub-volume. In one example, the third sub-volume comprises a part of the microfluidic channel separate to, i.e. not contained within the first sub-volume. In one example, the third sub-volume is contained entirely within the first-sub volume, i.e. the first and second capillary pressure barriers are both closed geometric configurations and the second capillary pressure barrier is completely encircled by the first capillary pressure barrier.

In some examples, the first and second capillary pressure barriers are both disposed on a base or floor of the microfluidic channel, or on the upper surface or ceiling of the microfluidic channel. In some examples, the first capillary pressure barrier defines a first sub-volume of the microfluidic channel aligned with the aperture. In some examples, the first and second capillary pressure barriers define a second sub-volume of the microfluidic channel concentric with the first sub-volume and aperture and enclosing the first sub-volume. In some examples, the first and second capillary pressure barriers are of a closed geometric configuration (e.g. circular) and the second capillary pressure barrier encircles the first capillary pressure barrier. In some examples, the first capillary pressure barrier comprises a first pair of linear capillary pressure barriers arranged on opposite sides of the aperture and extending to opposed inner surfaces to define the first sub-volume and the second capillary pressure barrier comprises a second pair of linear capillary pressure barriers arranged on opposite sides of the aperture, extending to opposed inner surfaces and spaced from and outside of the first capillary pressure to define a second sub-volume and a third sub-volume. In these examples, an external tissue sample, for example a tissue slice, or an organoid can be placed into the cavity created within and by the pinned gel or ECM, and more easily vascularised (once the gel has been vascularised) as it is in the same plane as the vascularised bed. This configuration also allows better positioning of the tissue for imaging of the whole system as all components are in the same focal plane.

### Reservoir

In some examples, the microfluidic network comprises a reservoir or well in fluid communication with a media inlet to the microfluidic channel. The reservoir may be present to retain a volume of liquid, for example culture media. In a typical embodiment the reservoir is able to retain a larger volume of fluid than is or can be retained by the microfluidic channel. The reservoir may be an adjacent well to the well aligned with the inlet aperture to the cell culture chamber on a bottomless microtiter plate disposed on top of the microfluidic layer. In other examples, the reservoir may be a well on the same microtiter plate, but spatially distant from the well of the strain compartment. It will be understood that the proximity of the reservoir to the well of the strain compartment is not critical to the operation of the device as long as the two are in fluid communication via the microfluidic layer.

In some examples, the microfluidic network comprises more than one, for example two, or more, reservoirs in fluid communication with the microfluidic layer and with the cell culture chamber or strain compartment and any other reservoir present in the microfluidic network. Each reservoir may be in fluid communication with the microfluidic layer via an aperture in the cover layer which may be termed an inlet, or an outlet, of the microfluidic layer as appropriate. In the embodiment in which at least two reservoirs are present in the microfluidic network, a first reservoir may be used for introducing a fluid, for example culture media into the microfluidic network, while the second reservoir may function as a vent, or overflow compartment for receiving the fluid during performance of the methods of the present invention.

In some examples, the microfluidic network of the device further contains biological or biomimetic material including one or more of:

a. gel, extracellular matrix or scaffold provided for example in the first sub-volume;

b. epithelial or endothelial cells lining the microfluidic channel and/or gel, for example forming a tube or blood vessel;

c. epithelial or endothelial cells situated inside, on or against a gel, extracellular matrix or scaffold, preferably forming lumened structures, more preferably forming a vascular bed;

d. stromal cells in, on or against a gel, extracellular matrix or scaffold;

e. muscle cells in, on or against a gel, extracellular matrix or scaffold;

f. one or more other cell types selected from pluripotent cells, central nervous, peripheral nervous, immune, urinary, respiratory, reproductive (male and female), gastrointestinal, endocrine, skin, musculoskeletal, cardiovascular, and mammary cell types.

Such devices may also be considered as assay plates due to the presence of the cells, for example in the form of a vascular network and the optional biological tissue disposed on a top surface of the extracellular matrix, thus being ready for use in assays or methods described herein. As will be understood from the present disclosure, the production of such devices may be realised using any of the methods described below. In one example, sprouts of endothelial cells extend into the extracellular matrix gel, forming a vascular bed. Optionally these sprouts are microvessels that are a result of angiogenesis or vasculogenesis.

The biological tissue in the form of any one or more of the above mentioned different cell types may comprise or be derived from healthy or diseased tissue, and may be obtained from or derived from a patient. The endothelial cells forming the vascular network may be obtained from or derived from a patient, for example the same patient from which the biological tissue has been obtained or derived. In one example, the endothelial cells comprise blood outgrowth endothelial cells (as for instance described in Nature Protocols 7, 1709-1715 (2012)) or endothelial cells derived from stem cells, including but not limited to induced pluripotent stem cells.

### Methods

In one example there is provided a method to assess mechanical strain induced by cells, comprising:
introducing one or more types of cells or cell aggregates into a microfluidic network of a microfluidic device described herein;
optionally culturing the one or more types of cells or cell aggregates; and
monitoring deflection of the diaphragm using one or more electrodes, sensors, probes, reference markers for monitoring diaphragm movement, ferromagnetic particles, or antibodies disposed on or operatively connected to the diaphragm.

In one example there is provided a method of subjecting one or more types of cells or cell aggregates to mechanical strain i.e. inducing mechanical strain in the one or more types of cells or cell aggregates, comprising:
introducing one or more types of cells or cell aggregates into the microfluidic network of a microfluidic device according to the first aspect;
optionally culturing the one or more types of cells or cell aggregates; and
subjecting the one or more types of cells or cell aggregates to mechanical strain by applying a positive pressure or a negative pressure to the diaphragm.

In some examples, the methods described herein comprise:
introducing into the microfluidic network a volume of a gel or gel precursor;
allowing the volume of gel or gel-precursor to cure or gelate to form a cured gel;
loading the microfluidic network with a fluid; and
culturing the one or more types of cells or cell aggregates.

In some examples, the methods described herein may comprise:
introducing a volume of gel or gel-precursor into the first sub-volume and allowing the volume of gel or gel-precursor to be confined by a capillary pressure barrier;
allowing the volume of gel or gel-precursor to cure or gelate to form a cured gel;
loading the microfluidic network with a fluid; and
culturing the one or more types of cells or cell aggregates.

In some examples, the volume of gel or gel-precursor may be a single droplet or droplet-sized volume of a gel or gel-precursor.

In some examples, following application of pressure to the diaphragm, the cellular response to the mechanical strain is monitored. In some examples, the cellular response may be from a monolayer of cells formed on an upper surface of a gel droplet or from a vascular bed formed within a gel. In some examples, the cellular response may be from a lumened cellular component contained within a microfluidic channel of a microfluidic device. In some examples, the cellular response may be from a lumened cellular component formed on the surface of the diaphragm.

The cellular response may be monitored in any way known in the art. Methods may include monitoring one or more of changes in pH, monitoring for changes in secreted factors (e.g. metabolites, growth factors, cytokines), sampling cells and/or tissues and monitoring up- or down-regulation of particular proteins, or monitoring levels of reactive oxygen species. Alternatively, or in addition, the cellular or tissue response may be monitored visually (using a microscope), for example based on immunohistochemical staining or other hybridization based staining.

In some examples, the one or more types of cells or cell aggregates may be selected from: epithelial or endothelial cells for lining the microfluidic channels, potentially forming a tube or blood vessel; epithelial or endothelial cells to be situated inside a gel, extracellular matrix or scaffold, preferably forming lumened structures, more preferably forming a vascular bed; stromal cells in or on a gel, extracellular matrix or scaffold; muscle cells in or on a gel, extracellular matrix or scaffold; one or more other cell types selected from pluripotent cells and central nervous, peripheral nervous, immune, urinary, respiratory, reproductive (male and female), gastrointestinal, endocrine, skin, musculoskeletal, cardiovascular, and mammary cell types.

The gel or gel-precursor includes any hydrogel known in the art suitable for cell culture. Hydrogels used for cell culture can be formed from a vast array of natural and synthetic materials, offering a broad spectrum of mechanical and chemical properties. For a review of the materials and methods used for hydrogel synthesis see Lee and Mooney (Chem Rev 2001; 101(7):1869-1880). Suitable hydrogels promote cell function when formed from natural materials and are permissive to cell function when formed from synthetic materials. Natural gels for cell culture are typically formed of proteins and ECM components such as collagen, fibrin, hyaluronic acid, or Matrigel, as well as materials derived from other biological sources such as chitosan, alginate or silk fibrils. Since they are derived from natural sources, these gels are inherently biocompatible and bioactive. Permissive synthetic hydrogels can be formed of purely non-natural molecules such as poly(ethylene glycol) (PEG), poly(vinyl alcohol), and poly(2-hydroxy ethyl methacrylate). PEG hydrogels have been shown to maintain the viability of encapsulated cells and allow for ECM deposition as they degrade, demonstrating that synthetic gels can function as 3D cell culture platforms even without integrin-binding ligands. Such inert gels are highly reproducible, allow for facile tuning of mechanical properties, and are simply processed and manufactured.

The gel precursor can be provided to the microfluidic cell culture device, for example to the strain compartment of a device as described above. After the gel is provided, it is caused to gelate, prior to introduction of a further fluid. Suitable (precursor) gels are well known in the art. By way of example, the gel precursor may be a hydrogel, and is typically an extracellular matrix (ECM) gel. The ECM may for example comprise collagen, fibrinogen, fibronectin, and/or basement membrane extracts such as Matrigel or a synthetic gel. The gel precursor may, by way of example, be introduced into the strain compartment with a pipette.

The gel or gel precursor may comprise a basement membrane extract, human or animal tissue or cell culture-derived extracellular matrices, animal tissue-derived extracellular matrices, synthetic extracellular matrices, hydrogels, collagen, soft agar, egg white and commercially available products such as Matrigel.

Basement membranes, comprising the basal lamina, are thin extracellular matrices which underlie epithelial cells in vivo and are comprised of extracellular matrices, such a protein and proteoglycans. In one example, the basement membranes are composed of collagen IV, laminin, entactin, heparan sulfide proteoglycans and numerous other minor components (Quaranta et al, Curr. Opin. Cell Biol. 6, 674-681, 1994). These components alone as well as the intact basement membranes are biologically active and promote cell adhesion, migration and, in many cases growth and differentiation. An example of a gel based on basement membranes is termed Matrigel (U.S. Pat. No. 4,829,000). This material is very biologically active in vitro as a substratum for epithelial cells.

Many different suitable gels for use in the method of the invention are commercially available, and include but are not limited to those comprising Matrigel rgf, BME1, BME1rgf, BME2, BME2rgf, BME3 (all Matrigel variants) Collagen I, Collagen IV, mixtures of Collagen I and IV, or mixtures of Collagen I and IV, and Collagen II and III), puramatrix, hydrogels, Cell-Tak™, Collagen I, Collagen IV, Matrigel® Matrix, Fibronectin, Gelatin, Laminin, Osteopontin, Poly-Lysine (PDL, PLL), PDL/LM and PLO/LM, PuraMatrix® or Vitronectin. In one preferred embodiment, the matrix components are obtained as the commercially available Corning® MATRIGEL® Matrix (Corning, N.Y. 14831, USA).

The gel or gel-precursor is introduced into a device described herein and confined by a capillary pressure barrier in the microfluidic device, for example to a first sub-volume of the network comprising a strain compartment having as its base a diaphragm of the device, and then caused or allowed to gelate.

In one example, a droplet of a sufficient volume is introduced such that the cured gel is located substantially entirely within the part of the strain compartment that is within the microfluidic layer. In one example, the volume of gelled droplet is such that the droplet does not fully block the aperture in the microfluidic cover layer, in which case the unblocked or open region of the aperture can be used as a vent. A vent thus generally comprises an opening or aperture in the cover layer allowing evacuation of air when loading the microfluidic channel through the inlet. In one example, a droplet of a sufficient volume is introduced such that the droplet is confined by the capillary pressure barrier and the majority of the droplet volume is contained within the part of the strain compartment that is outside of the microfluidic layer, for example wherein the majority of the droplet volume is contained within the well of the top layer.

In one example, the gel or gel-precursor is preloaded with the cell or cells of interest, i.e. the cells are present in the droplet of gel or gel-precursor prior to introduction into the microfluidic cell culture device, and prior to gelation. In another example, the cells are inserted into the partially or fully cured droplet after it has been introduced into the microfluidic cell culture device, for example to a strain compartment of a device described herein. Thus, an alternative method comprises seeding the cured droplet of cell culture hydrogel with the cells of interest. In another example, the gel or gel-precursor is introduced into the microfluidic cell culture device, and following gelation, cell mixture, tissue or cell aggregate is placed on top of the gel or into a region of the microfluidic channel adjacent to the gel.

The cell mixture, tissue or cell aggregate in, on or alongside a cured gel may include epithelial or endothelial cells, stromal cells, muscle cells, one or more other cell types selected from pluripotent cells and central nervous, peripheral nervous, immune, urinary, respiratory, reproductive (male and female), gastrointestinal, endocrine, skin, musculoskeletal, cardiovascular, and mammary cell types.

In one example, the at least one type of cell or cell aggregate present in or on top of the droplet of gel or gel-precursor comprises epithelial cells, which during culture can proliferate and/or differentiate depending on the composition of the culture media, other cell types which may be present, and the extracellular matrix. Thus, after introduction into the microfluidic network, either using an aqueous medium, preferably a growth medium, or by using the gel (precursor), the epithelial cells are then allowed to proliferate and/or differentiate. Culture of the one or more types of cells or cell aggregates, for example epithelial cells, is achieved by introduction of media into the microfluidic channel and continued under suitable conditions so that the cells are cultured. For the avoidance of doubt, use of the term “droplet” is not to be construed as meaning that the gel has a typical droplet form or shape. Instead, it is to be construed as meaning the volume of gel that is introduced into and then confined within the cell culture devices described herein.

In one example, following gelation of a gel-precursor in a first sub-volume of the microfluidic network, for example a strain compartment, one or more cells or cell aggregates are introduced into a second sub-volume of the microfluidic network, for example a region of the microfluidic channel adjacent to the gel and the capillary pressure barrier. The one or more cells or cell aggregates may be epithelial cells or endothelial cells. In general endothelial cells are known as the cells that line the interior surface of the entire circulatory system, from the heart to the smallest lymphatic capillaries. When in contact with blood these cells are called vascular endothelial cells and when in contact with the lymphatic system they are called lymphatic endothelial cells. In a particular embodiment the culture method includes the step of introducing endothelial cells into the microfluidic channel of the microfluidic network, and causing or allowing said endothelial cells to line the microfluidic channel, i.e. causing or allowing the endothelial cells to form a vessel within the microfluidic channel. The cells or cell aggregates may be introduced into the microfluidic network using any suitable medium.

Introducing endothelial cells into the microfluidic channel under the right conditions, for example conditions suitable to promote angiogenesis, can result not only in the formation of vascular tissue lining the internal surfaces of the microfluidic channel and in some cases the internal surfaces of the extracellular matrix gel which then becomes permeable, but also outgrowth of new microvessels. The conditions suitable to promote angiogenesis include adding pro-angiogenic compounds such as Fibroblast growth factor (FGF), Vascular Endothelial Growth Factor (VEGF), Angiopoietin-1 (Ang1), Angiopoietin-2 (Ang2), phorbol myristate-13-acetate (PMA), Sphingosine-1-phosphate (S1P), IGFBP-2, hepatocyte growth factor (HGF), prolyl hydroxylase inhibitors (PHi), monocyte chemotactic protein-1 (MCP-1), basic fibroblast growth factor (bFGF) and ephrins amongst others.

When applied as a gradient, the one or more pro-angiogenic compounds can be considered to act as a chemoattractant that promotes directional angiogenesis toward and within the confined gel droplet. In this way, the endothelial cells are stimulated to form a layer of vascular tissue in the microfluidic layer and in the gel which then undergoes permeabilisation and results in outgrowth of new microvessels. The one or more proangiogenic compounds may be added to the droplet of gel or gel-precursor before it is introduced into the microfluidic network, or it may be added to after formation of the gel, for example onto the top surface of the gel. In another example, the one or more proangiogenic compounds may be added to the microfluidic network via another inlet into the microfluidic channel, for example an inlet downstream from the inlet through which the culture media is introduced and/or downstream from the strain compartment.

In some examples, following formation of vascular tissue in the microfluidic layer and gel, the methods may further comprise introducing one or more types of cells, preferably including at least one type of epithelial cells, to a third sub-volume of the microfluidic network, via an inlet aperture; and allowing the one or more types of cells to form a (mono-)layer or cell aggregate. For example, the one or more types of cells may form a monolayer of cells on top of a gel confined to the first sub-volume.

In one embodiment, the one or more cells, or cell aggregates, fully cover the top surface of the at least partially cured gel, thereby forming a barrier layer of tissue on the top surface of the at least partially cured gel. The barrier layer may comprise a monolayer of cells, or a multi-layer of cells or cell aggregates. In one embodiment, the monolayer of cells or the multilayer may be cultured, to allow proliferation and/or differentiation, before or after angiogenesis of the at least one microvessel into the at least partially cured gel. Examples of flat layered tissue include skin tissue (comprising e.g. keratinocytes, adipose tissue and fibroblasts), gut epithelium as well as other epithelial tissues such as lung and retina.

Culture media, or differentiation media may be added to the microfluidic channel as described above, and establishment of a fluid flow through the vascular network may also be achieved as described above, to allow for cell proliferation and/or differentiation. Similarly, compositions of fluids can be controlled as described above. Thus, the vascularised, perfusable network established by the method described allows for the free exchange of metabolites, nutrients and oxygen between the fluid in the microvessel within the microfluidic channel of the device and the cells or cell aggregates on top of the cured gel.

As already explained herein before, using the capillary pressure barriers enables the formation of stable confined volumes of gel, for example, in sub-volumes of the microfluidic network so that addition of a second fluid can take place without displacing the gel or its contents. The device of the present invention is thus configured for spatially controlled co-culture with other cells as described above, and provides means to control the composition of the surrounding medium. As such, and within the methods of the present invention a fluid loaded into a reservoir (herein also referred to as a well) is any of cell culture media, test solutions, buffers, further hydrogels and the like and may optionally comprise cells or cellular aggregates.

By controlling the composition(s) introduced in the reservoir(s) the cell culture device of the present invention enables different modes of cell culture. For example, the composition of fluids introduced into the reservoirs or wells can be changed. Such exchange can be a gradient exchange by introducing a new composition in one of the reservoirs and simultaneously removing the fluid from another reservoir within the same microfluidic network till complete exchange has occurred. Such exchange can be discrete, by aspirating fluid from the reservoir and filling it with the new composition. The fluid volume in the reservoir is much larger than the fluid volume in the microfluidic channel and the levelling between reservoirs occurs almost instantaneously, thereby assuring flushing the microfluidic network with the new fluid without the need for emptying the microfluidic channel network during the procedure.

In one example, presence of a second capillary pressure barrier in the device even allows the formation of a layered gel composition. In this example, a first capillary pressure barrier, for example a circular capillary pressure barrier pins a liquid composition comprising a first gel or gel-precursor as a standing droplet on the base layer of the microfluidic network, for example on the diaphragm. After this first liquid composition is set, a second gel or gel precursor, optionally containing cells, is loaded. This second composition will be retained by a second capillary pressure barrier, for example a circular capillary pressure barrier of larger diameter than the first capillary pressure barrier and concentric with and encircling the first capillary pressure barrier. Through this configuration, the second capillary pressure barrier prevents this second composition from flowing into the microfluidic channel and encapsulates the first gel. The presence of the two capillary pressure barriers accordingly divides the microfluidic network into individual spatial volumes, and gives the user the possibility of spatial configuration in the microfluidic network.

The discussion on the methods thus far has described how cells or cell aggregates may be incorporated into a microfluidic device as described herein. Once a device has been loaded with cells or cell aggregates and any necessary culturing of the cells has taken place, the methods may comprise one or more steps of subjecting the cells to mechanical strain and/or measuring mechanical strain emanating from the cells.

A step of subjecting the cells to mechanical strain may comprise applying a positive or negative pressure, in one example an alternating positive pressure and negative pressure, to the diaphragm. Application or pressure will induce deformation of the diaphragm into the microfluidic channel (in the case of a positive pressure applied from underneath the diaphragm) or away from the microfluidic channel into the base layer (in the case of a negative pressure applied from underneath the diaphragm). In these methods, the surface of the diaphragm facing into the microfluidic channel may have one or more cells or cell aggregates directly disposed on it. In addition or alternatively, the one or more cells or cell aggregates may be disposed on a surface of the microfluidic channel in proximity to the diaphragm, for example lining a surface of the microfluidic channel. The one or more cells or cell aggregates may also be disposed in or on a gel confined to the surface of the diaphragm by one or more capillary pressure barriers in the microfluidic channel. The one or more cells or cell aggregates are generally disposed at a location within the microfluidic network at which displacement of the diaphragm can still have an effect. It will be appreciated that the further the cells or cell aggregates are from the diaphragm, the greater the displacement will be required in order to exert an effect over the cells. Thus, in one example, the one or more cells or cell aggregates are present in or on a gel disposed on a surface of the diaphragm facing the microfluidic channel.

In some examples of the methods described herein, mechanical strain is varied through time by displacing the diaphragm in a single, cyclical or repeating pattern. That is, the diaphragm may be displaced a plurality of times, in a particular rhythm or sequence. For example, the diaphragm may be displaced in a rhythmic manner similar to breathing, to recreate mechanical strain in lung tissue. In another example, the diaphragm may be displaced in a manner to recreate peristaltic movement of intestinal tissue.

Ways of displacing or deforming a diaphragm in a microfluidic device are known in the art, and have been discussed above in connection with the microfluidic device. In some examples, the device may comprise a plurality of diaphragms in contact with the microfluidic channel. The plurality of diaphragms may be configured such that multiple actuations of one or more of the plurality of diaphragms in a predetermined pattern causes a net fluid movement through the microfluidic network over the course of multiple actuation cycles.

In some examples, displacement of the diaphragm is to such an extent that mechanical strain can be applied to a monolayer of cells on the upper surface of a gel present on an upper surface of the diaphragm. As described above, mechanical strain can be applied to such a monolayer of cells by application of positive or negative air pressure, or by application of a force from a mechanical actuator.

In some examples of the methods described, displacement of the diaphragm is not actuated externally, and is instead caused by or induced by one or more cells or cell aggregates present in the microfluidic layer, for example present on the diaphragm, for example in or on a gel or ECM on the diaphragm. The one or more cells or cell aggregates may also be disposed directly on the diaphragm, optionally aided by a coating of cell adhesion molecules on the diaphragm. In these examples, the diaphragm is advantageously functionalised with one or more electrodes, sensors, or reference markers for monitoring diaphragm movement.

In one example, markers are imprinted into the same material of the diaphragm, i.e. by etching, milling, or by including the markers in a mould with which the membrane is formed. In another example, markers, sensors or transducers may be added to the diaphragm material, e.g. by adding it to the material during manufacturing. For example, magnetic beads could be mixed with the polymer(s) making up the diaphragm, that are subsequently used for actuation or sensing. Alternatively, a coil could be embedded in the polymer(s). In yet another example, material is applied to the diaphragm by surface deposition of said material, for example sputtering, plasma deposition, screen printing, or other forms of printing or deposition. Such processes could be used to print markers from ink, metals or other materials that can be used to monitor deflection.

### Assay Plate

A further aspect of the present invention provides an assay plate, comprising any of the devices described herein. References to cell culture devices comprising a vascular network and optionally also a biological component such as a monolayer of cells are to be understood as also referring to an assay plate.

In one example there is provided an assay plate, comprising a microfluidic device as described herein with a gel confined by the capillary pressure barrier to a first sub-volume of the microfluidic channel, wherein the microfluidic network comprises one or more cells or cell aggregates, present for example in or on the gel, and/or in a microfluidic channel.

The assay plate may comprise one or more cells or cell aggregates which have been cultured by the methods described herein. In one example, at least a part of a microfluidic channel of the device of the assay plate comprises a layer of vascular tissue comprising endothelial cells extending into the gel.

The dimensions of the assay plate may be consistent or compatible with the standard ANSI/SLAS microtiter plate format. In particular the dimensions of the footprint or circumference of the assay plate may be consistent with the ANSI/SLAS standard for microtiter plates.

Also described are assay plates, or cell culture devices produced by any of the methods described herein.

### Kits

The present disclosure also provides kits and articles of manufacture for using the microfluidic devices and assay plates described herein. In one embodiment, the kit comprises the devices or assay plates described herein; and one or more pro-angiogenic compounds, for inducing angiogenesis. In some examples, the kit may comprise the device or assay plate described herein and one or more of: a gel, gel-precursor composition or other extra-cellular matrix composition; one or more cells or cell types; growth media; and one or more reagent compositions, including one or more pro-angiogenic compounds.

The cell culture device or assay plate of the kit preferably comprises a vascular bed, in other words comprises an extracellular matrix gel arranged to receive at least one cell to be vascularised on a top surface thereof; and a vascular network of endothelial cells lining the internal surfaces of the microfluidic channel.

The kit may further comprise a packaging material, and a label or package insert contained within the packaging material providing instructions for inducing angiogenesis in the cell culture device or assay plate using the one or more pro-angiogenic compounds.

The one or more proangiogenic compounds may comprise one or more of Fibroblast growth factor (FGF), Vascular Endothelial Growth Factor (VEGF), Angiopoietin-1 (Ang1), Angiopoietin-2 (Ang2), phorbol myristate-13-acetate (PMA), Sphingosine-1-phosphate (S1P), IGFBP-2, hepatocyte growth factor (HGF), prolyl hydroxylase inhibitors (PHi). monocyte chemotactic protein-1 (MCP-1), basic fibroblast growth factor (bFGF) and ephrins amongst others.

The kits may further include accessory components such as a second container comprising suitable media for introducing the one or more pro-angiogenic compounds, and instructions on using the media.

The present invention will now be described by way of example only, with reference to the drawings.

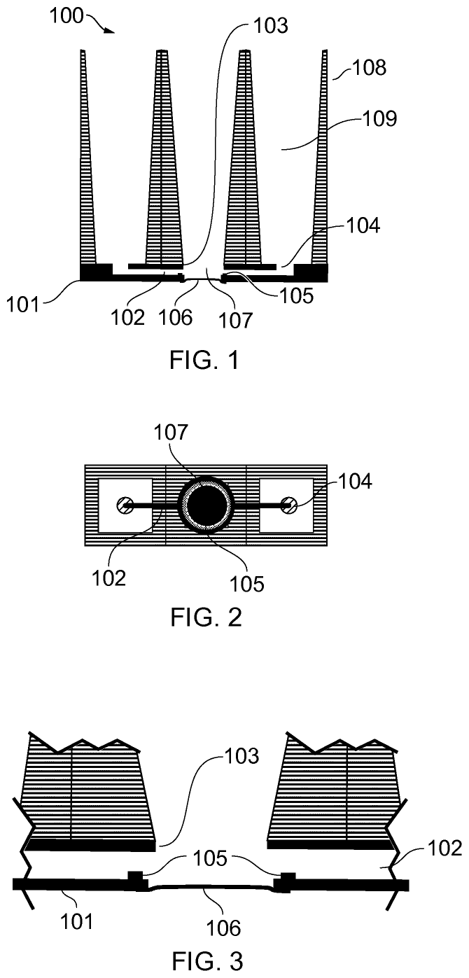

A first example of a microfluidic device is schematically shown in FIGS. 1 to 3. The device (100) as shown in FIG. 1 generally comprises a base (101), a microfluidic channel (102) in a microfluidic layer and a cover (103) (all shown in solid). Media inlets (104) are present in the cover layer of the microfluidic layer. A capillary pressure barrier (105) is present on the base (101) of the device and accessible via aperture (107) in the cover layer (103). In this particular example, base (101) is also provided with an aperture, across which a diaphragm (106) extends. A top layer (108) in the form of a multiwell bottomless plate is disposed on top of the cover layer and includes wells (109) positioned above each of inlet aperture (107) and media inlets (104).

As shown in the top view (FIG. 2), the circular capillary pressure barrier divides the microfluidic network in two sub-volumes. One sub-volume, in this embodiment the central volume within the capillary pressure barrier, comprises the strain compartment or cell culture chamber, and the second sub-volume defined by microfluidic channel (102) leading to and surrounding the first sub-volume. Microfluidic channel (102) is schematically represented in FIG. 2 as the solid circle surrounding the capillary pressure barrier, with the aperture (107) indicated by the dotted line.

The formation of two sub-volumes in direct contact with one another without any intervening structure such as a wall or membrane is one of the key characteristics of the device and assay plate. In addition, through the wells and the microfluidic channel it is even possible to control and adapt the medium surrounding the gel, for example, before or during strain experiments. FIG. 3 provides a close up view of the vertical cross section of a part of the microfluidic network, showing the capillary pressure barrier (105) on the base layer and diaphragm (106). The presence of the capillary pressure barrier (105) prevents a gel or gel precursor, for example from filling the microfluidic channels when loaded from above—in other words, in-use the gel or gel precursor is pinned on the capillary pressure barrier (105). FIG. 3 also shows one possible configuration of attaching a diaphragm to the base layer, namely by fixing the diaphragm to the lower surface of the base layer.

The aperture (107) in FIG. 2 as well in subsequent figures is depicted as a circular shaped aperture. However, it will be understood that the aperture can have any shape, with circular and square being preferred.

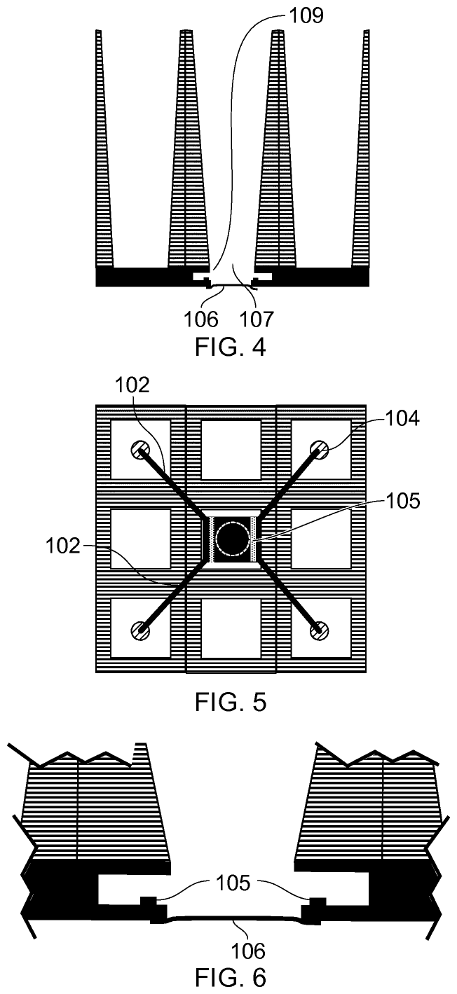

Being an objective to control the medium composition surrounding a gel composition confined by a capillary pressure barrier, additional branches of the microfluidic channel (102) may be present. One such example is provided in FIGS. 4 to 6. In this embodiment a central strain compartment is connected to four media inlets (104) in a cross configuration (see FIG. 5), with two linear capillary pressure barriers (105) present, each defining in part the first sub-volume comprising the strain compartment.

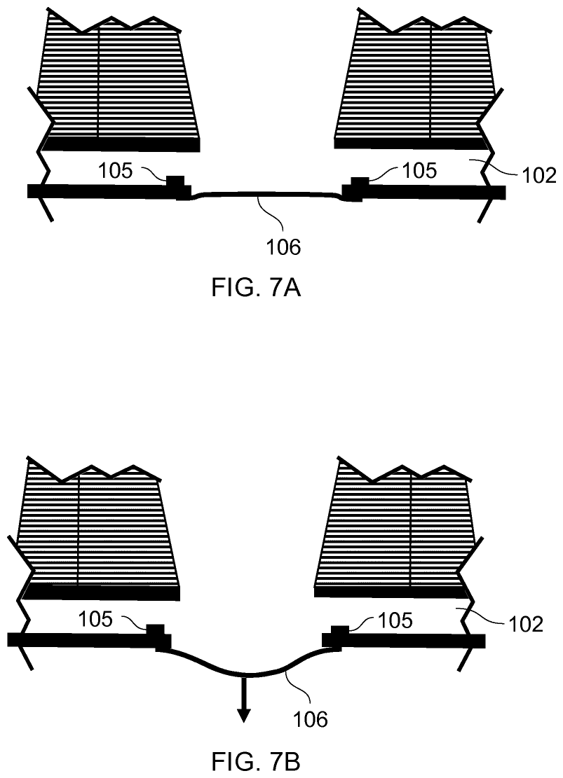

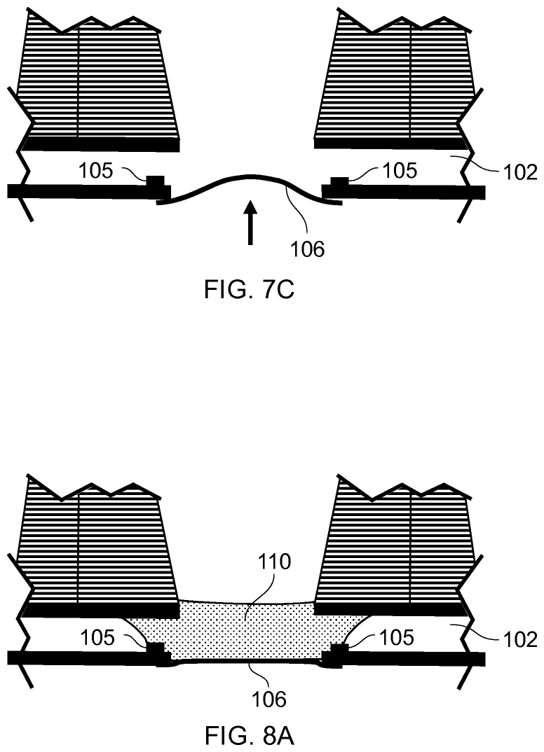

FIGS. 7A to 7C show the various states in which the diaphragm can exist before, or during a strain experiment. Generally, the diaphragm exists in a relatively taut state even when at rest, as shown in FIG. 7A. FIG. 7B shows the diaphragm in a strained state, and deflecting away from microfluidic channel 102 upon application of a negative pressure from below the diaphragm, such as might be applied using a vacuum pump. However, it will be understood that the same effect could also be achieved by application of a positive pressure from above the diaphragm. FIG. 7C shows the diaphragm in an alternative strained state, and deflecting into microfluidic channel 102 upon application of a positive pressure from below the diaphragm, such as might be applied using a pump, a mechanical actuator such as a pin, or an expandable foam. However, it will be understood that the same effect could also be achieved by application of a negative pressure from above the diaphragm.

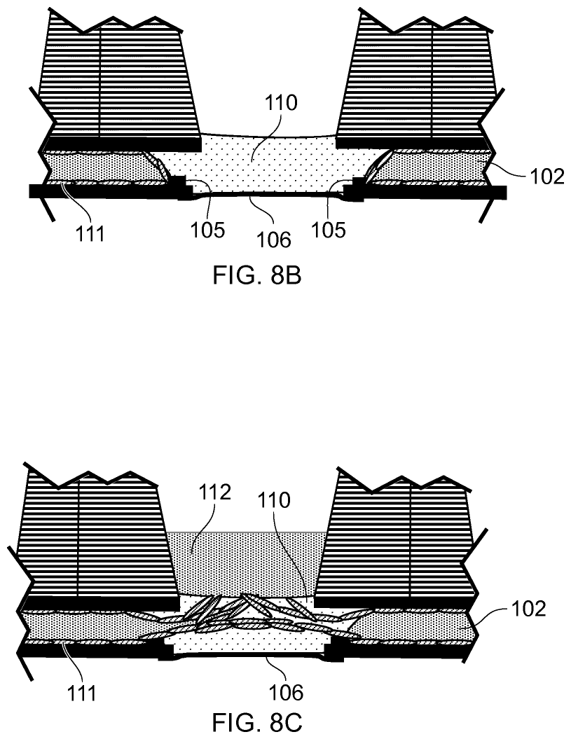

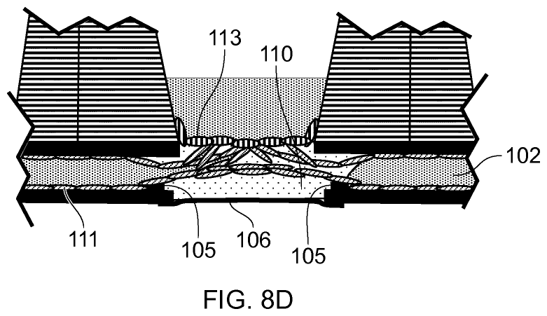

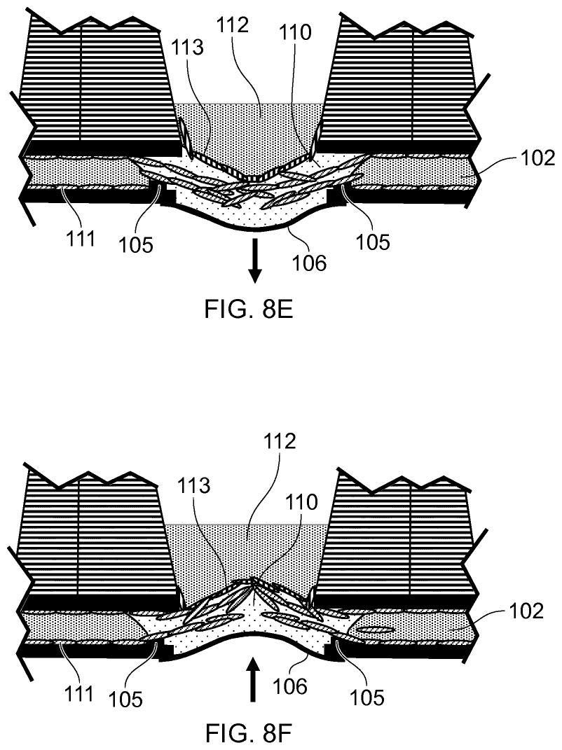

The different steps in a method using the device described herein is shown in FIGS. 8A to 8F. In a first step, a first droplet of gel or gel precursor (110) is introduced, pinned on the capillary pressure barrier and allowed to set (cure, gelate). Again, and as already mentioned hereinbefore, the first liquid composition will typically comprise a gel or gel-precursor, for example a hydrogel (or precursor thereof) used for cell culture and includes any hydrogel known in the art and suitable for the purpose. The gel may optionally comprise a suspension of cells.

Once the droplet of gel is set, the microfluidic channel is loaded with a second liquid so that endothelial cells (111) are introduced into the microfluidic channel (FIG. 8B).

These may be introduced as a component of a cell culture or growth media, or may be introduced subsequently. Upon culture of the thus seeded device, and dependent on the composition of the second liquid (the liquid loaded in the microfluidic channel), the endothelial cells (114) may vascularise or line the internal surfaces of the microchannel, i.e. the walls, base and top, and potentially also the ECM gel surfaces.

In a further step, addition of a fluid (112) including pro-angiogenic agents to the top of gel (110) could allow or induce angiogenesis of the vessels formed in the microfluidic channel (FIG. 8C), with invasion of the gel droplet and/or capillary vessel formation therein to form a vascular bed. Culture conditions allowing angiogenesis are known to the skilled artisan and include for example deprivation of oxygen, mechanical stimulation and chemical stimulation using pro-angiogenic agents such as the pro-angiogenic proteins described previously.

A typical spouting mixture comprises VEGF, MCP-1, HGF, bFGF, PMA, S1P in amounts of 37.5 ng/ml to 150 ng/ml for each of VEGF, MCP-1, HGF, bFGF and PMA, and 250 nM to 1000 nM for S1P. An alternative typical sprouting mix composition comprises S1P 500 nM, VEGF 50 ng/ml, FGF 20 ng/ml, PMA 20 ng/ml.

In this way, a vessel is formed that connects the inlet and outlet of the microfluidic channel, lines the channel surfaces and extends into the gel.

The preferable result of this method is a gel that comprises a vascular bed of microvessels that connect to a larger vessel via one or more microfluidic channels through which a flow of growth medium, serum or other can be applied. As such, and using the device, it is possible to co-culture a first type of cells in a first confined sub-volume of the network comprising the gel with culture of endothelial cells in the second sub-volume comprising the microfluidic channel, to achieve a vascularized model of the cellular aggregates present within the gel droplet or on top of the gel droplet, which is connected to the reservoir(s) by means of the endothelial vessels formed within the microfluidic channels.

It is not essential to use a cocktail of angiogenic compounds in order to achieve a vascular bed or vascularised tissue. In an alternative method in generating a vascular bed, a tissue is placed on top of the gel. The tissue itself excretes factors that induce angiogenesis, resulting in sprouting of the main vessel and formation of a vascular bed or even a vascularised tissue.

FIG. 8D shows addition of cells (113) to the top of the gel (110), which are then allowed to form a monolayer. The cells may be of any type, but would typically be epithelial or endothelial cells, depending on the strain experiments being performed. Finally, FIGS. 8E and 8F show the bidirectional deformation of diaphragm (106) during a strain experiment, leading to consequential application of strain to the monolayer of cells (114) on top of the gel comprising the microvessels (110).

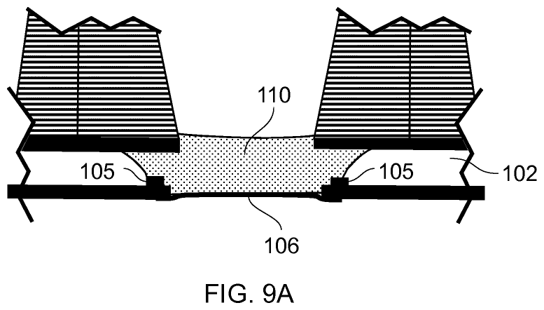

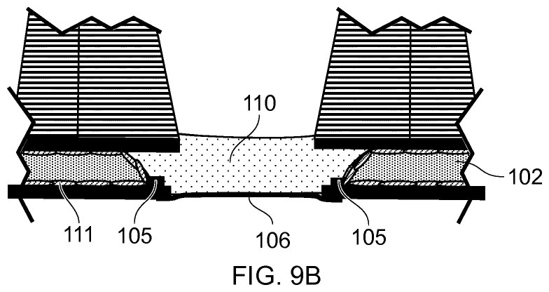

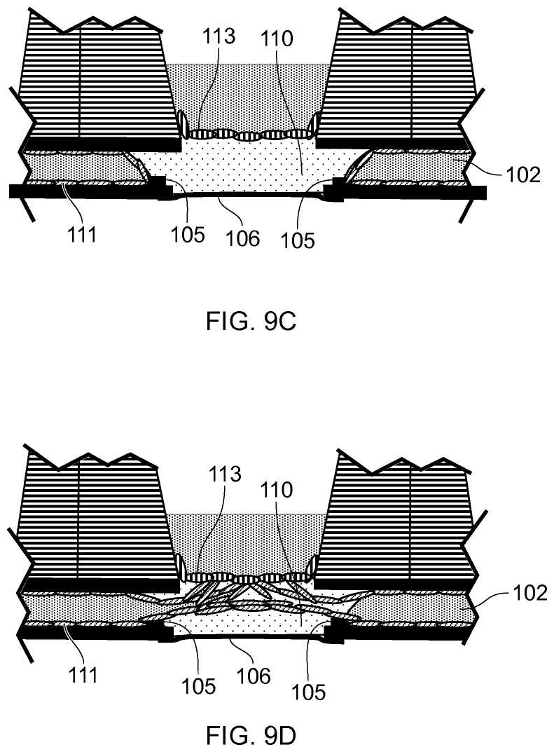

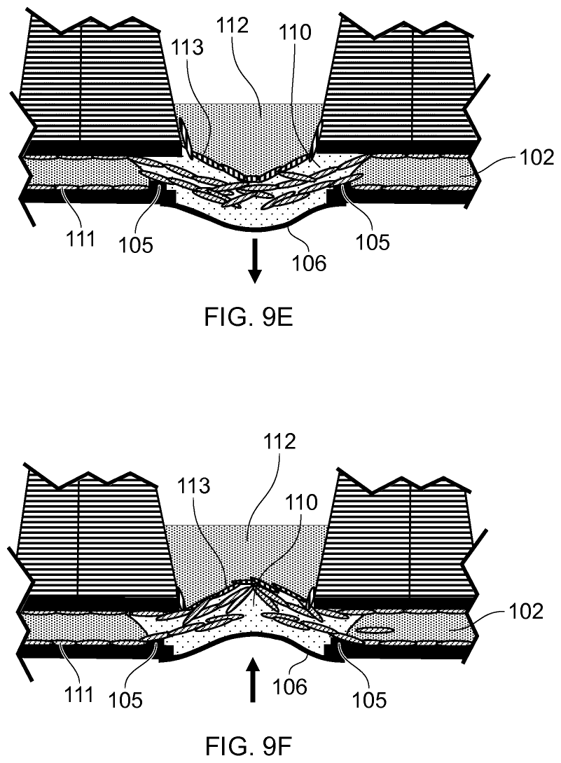

FIGS. 9A to 9F depict an alternative method to that depicted in FIGS. 8A to 8F. In this alternative method, the first two and last three steps are identical to the method of FIG. 8, with the only difference being the order in which (i) the cells (113) are added to the surface of gel (110) and (ii) vascularisation of gel (110) by endothelial cells (111) takes place.

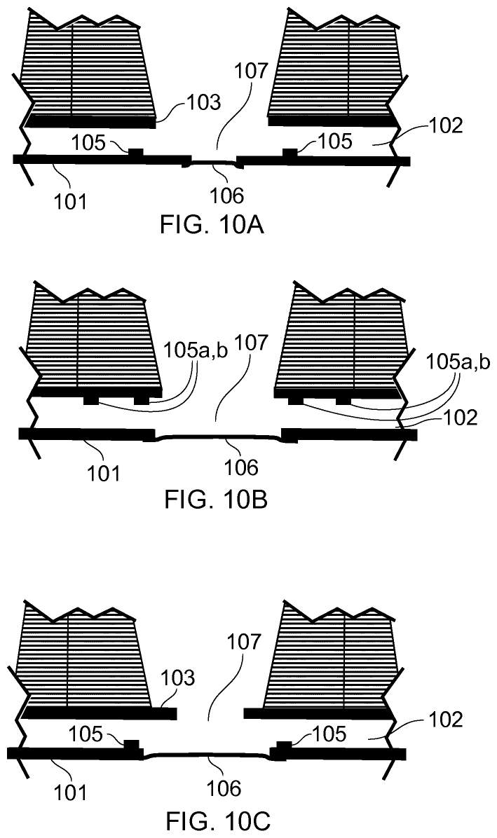

FIGS. 10A to 100 show close up vertical cross-section views of alternative configurations for a microfluidic network. Specifically, FIG. 10A shows an aperture in base (101) across which diaphragm (106) extends, with capillary pressure barrier (105) outside of the aperture. In this particular configuration, diaphragm (106) is aligned with but inlet aperture (107) but the exposed or available surface of diaphragm (106) is narrower than the cross-section of inlet aperture (107), while capillary pressure barrier (105) is distanced from diaphragm (106) and outside of inlet aperture (107). In contrast, in the configuration of FIG. 10B, the aperture across which diaphragm (106) extends is broadly of the same dimension as inlet aperture (107) such that the exposed or available surface area of diaphragm (106) is broadly of the same dimension as the cross-sectional area of inlet aperture (107), but with two capillary pressure barrier (105a,b) located on underside of cover (103). Finally, in FIG. 100, the aperture across which diaphragm (106) extends is larger than inlet aperture (107) such that the exposed or available surface area of diaphragm (106) is larger than the cross-sectional area of inlet aperture (107), with capillary pressure barrier (105) immediately adjacent diaphragm (106). Generally speaking, the larger diaphragm and/or the larger aperture, the more of the epithelium can be exposed to mechanical strain. In contrast, a smaller diaphragm will (for the same applied force) not displace as much as a larger diaphragm and so will lead to less displacement and/or damage to the strained tissue, particularly around the region of the edges of the inlet. It will be understood that the only general requirement is that the diaphragm is substantially aligned with the inlet aperture for ease of introduction of material into the device.

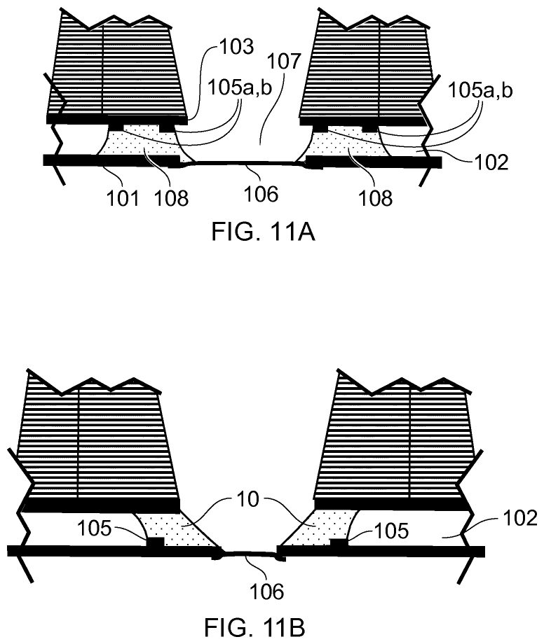

FIGS. 11A and 11B respectively show a gel or extracellular matrix (108) pinned by capillary pressure barriers (105a,b) and the rims of apertures (107) of the configurations of microfluidic networks as depicted in FIG. 10B and FIG. 10A. In these configurations, the gel is not pinned by any capillary pressure barrier so as to be disposed on the diaphragm to any meaningful extent and is instead pinned predominantly within the microfluidic channel. In these configurations an external tissue sample, for example a tissue slice, or an organoid can be placed into the cavity created within and by the pinned gel or ECM and more easily vascularised (once the gel 108 has been vascularised) as it is in the same plane as the vascularised bed. This configuration also allows better positioning of the tissue for imaging of the whole system as all components are in the same focal plane.

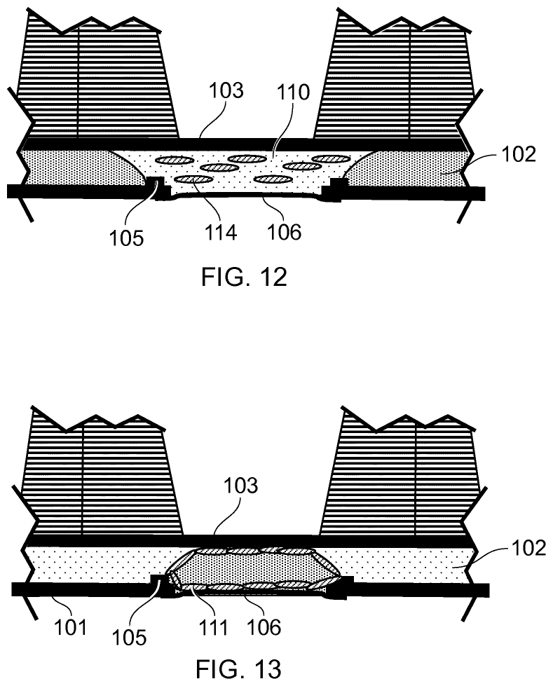

FIGS. 12 and 13 show uses of an alternative configuration of a microfluidic network as used in a device, specifically one in which there is no aperture in cover (103) aligned with diaphragm (106). FIG. 12 depicts a set-up for measuring mechanical strain or movement emanating from cells (114), for example contraction of muscle cells, fibroblasts, cardiomyocytes, or for measuring induced pressure on brain cells, bone cells, or compression of other biological tissues. FIG. 13 shows an alternative use of this particular configuration, in which cells (111) are allowed to form a lumened structure on the diaphragm, for example around a gel pinned by capillary pressure barrier (105). The cells may comprise endothelial cells forming a blood vessel, epithelial cells forming an intestinal type lumen or a kidney tubule type lumen, or cardiomyocytes forming an atrial or ventricular type lumen. Devices of this type thus allow the monitoring or induction of mechanical strain resulting from or mimicking vasodilation/vasoconstriction, gut peristaltic motion, kidney tubule compression, vascular compression and cardiomyocyte actuation, or indeed inducing such activities as the case may be.

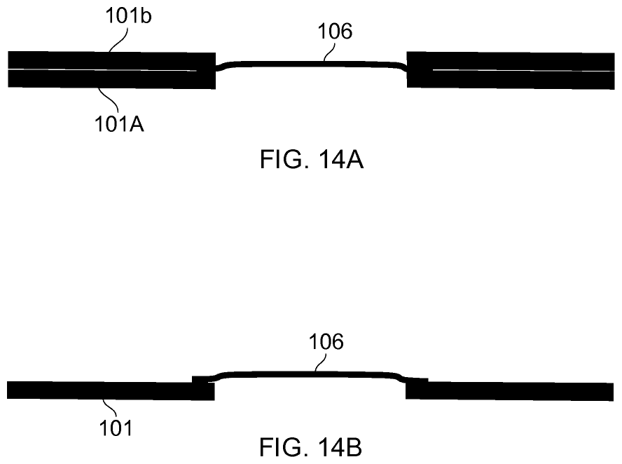

FIGS. 14A and 14B show alternative ways of fixing a diaphragm to the base of a microfluidic network or device as herein described. FIG. 14A shows diaphragm (106) clamped between two sub-layers (101a, 101b) of a base layer, while FIG. 14B shows diaphragm (106) fixed to an upper surface of base layer (101).

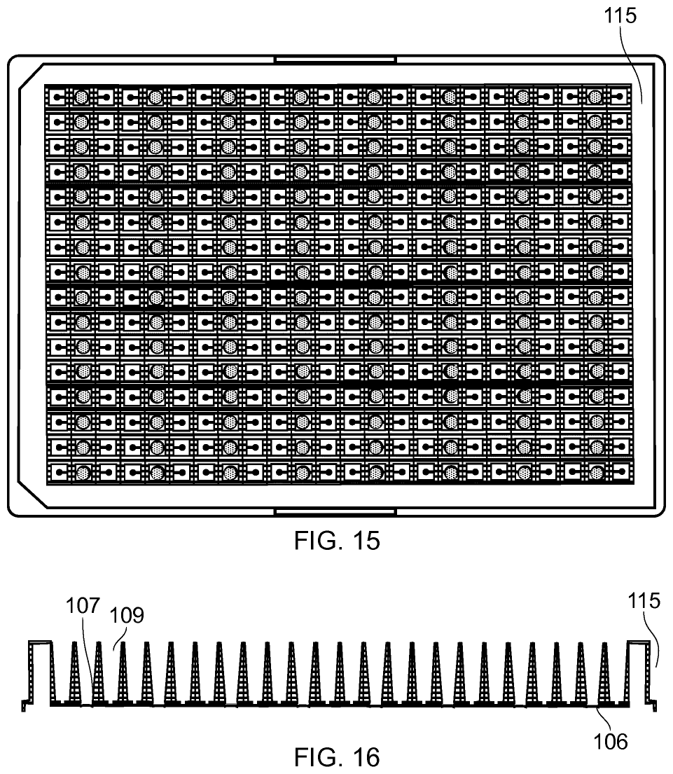

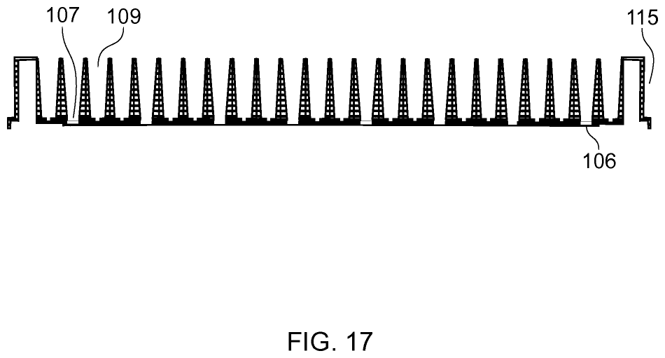

FIG. 15 shows a plan view of a multi-well device (115) according to the invention and consisting of a multi-well configuration of the microfluidic networks as herein described. As described, the device is preferably compatible with or based on a microtiter plate footprint as defined by ANSI/SLAS dimensions, as shown in FIG. 15, which shows a bottom view of such a plate comprising 128 separate microfluidic networks such as for example described in FIG. 1. In the centre of each microfluidic network a diaphragm (106) is indicated. FIG. 16 shows a cross-section of the multi-well configuration of FIG. 15, with an individual diaphragm extending across an aperture in each microfluidic network. FIG. 17 shows an alternative configuration to that of FIG. 16, with a single sheet of elastomer extending the entire width of device (115), thus extending across the individual apertures of the independent microfluidic networks.

### Examples

A method of constructing a base layer for use in a device will now be described.

Generally, the base layer comprises two sheets of milled glass each with a plurality of apertures of 2 mm in diameter, and a flexible diaphragm, for example a polyurethane diaphragm. The diaphragm is placed between the two sheets of glass, and the glass sheets aligned such that the apertures are aligned. The three layers are then placed under heat and pressure, at 4 bar and 95° C. The finished product is a base layer for a microfluidic device, the base layer consisting of two pieces of milled glass laminated with a polyurethane diaphragm which can provide actuation to a microfluidic channel of a microfluidic network.

The base layer was connected to a manifold which comprised a 10 mm thick sheet of polycarbonate and a silicone gasket connected to a pressurized air supply. A pressure of 1 bar was applied. Visual investigations under a microscope and/or photography confirmed displacement of each diaphragm to which pressure was applied.

The above description is for the purpose of teaching the person of ordinary skill in the art how to practice the present invention, and it is not intended to detail all those modifications and variations which will become apparent upon reading the description. It is intended, however, that all such modifications and variations be included within the scope of the present invention, which is defined by the following claims.

1. A microfluidic device, comprising:
a microfluidic network, the microfluidic network comprising:
a base, a microfluidic channel, and a cover;
wherein the base comprises a non-porous diaphragm forming at least part of an inner surface of the microfluidic channel and wherein the microfluidic channel comprises a sub-volume defined at least in part by the diaphragm and by a capillary pressure barrier in the microfluidic channel.

2. The microfluidic device of claim 1, wherein the diaphragm comprises an elastomer.

3. The microfluidic device of claim 1, wherein the cover comprises an inlet aperture to the microfluidic channel and wherein the inlet aperture is substantially aligned with the diaphragm.

4. The microfluidic device according to any one of the preceding claims, wherein the base comprises an aperture to the microfluidic channel across which the diaphragm extends.

5. The microfluidic device according to any one of claims 1 to 4, wherein the diaphragm comprises a region of the base which is of thinner cross-section than the surrounding portion of the base.

6. The microfluidic device according to any one of the preceding claims where the diaphragm is transparent or optically clear and preferably has a thickness of less than 1 mm, more preferably less than 250 μm, more preferably less than 100 μm.

7. The microfluidic device according to any one of the preceding claims, wherein the diaphragm is a functionalised diaphragm comprising one or more electrodes, sensors, probes, reference markers for monitoring diaphragm movement, ferromagnetic particles, adhesion molecules, or antibodies.

8. The microfluidic device according to any one of the preceding claims, wherein the sub-volume is a first sub-volume and wherein the microfluidic channel further comprises:
a second sub-volume comprising a flow channel; and
a third sub-volume that is separated from the second sub-volume by the first sub-volume.

9. The microfluidic device according to any one of claims 2 to 8, wherein the device further comprises a top layer having a well and wherein the first and/or third sub-volume extends into the well via the inlet aperture.

10. The microfluidic device according to any one of claims 2 to 9, wherein the capillary pressure barrier is substantially aligned with the inlet aperture.

11. The microfluidic device according to any of claims 1 to 10, wherein the diaphragm forms at least part of the surface of the first sub-volume.

12. The microfluidic device according to any of claims 1 to 11, wherein the diaphragm forms at least part of the surface of the third sub-volume, the third sub-volume being optionally confined by a further capillary pressure barrier.

13. A microfluidic device according to any one of the preceding claims, wherein the base is configured to operatively connect the diaphragm to one or more of:
a source of positive or negative (air-)pressure;
a physical actuator;
an electromagnetic actuator; and
an expandable foam.

14. The microfluidic device according to any of the preceding claims, wherein the capillary pressure barrier comprises:
a ridge of material protruding from an internal surface of the microfluidic channel;
a widening of the microfluidic channel;
a groove in an internal surface of the microfluidic channel;
a region of material of different wettability to an internal surface of the microfluidic channel; or
a plurality of pillars at regular intervals.

15. The microfluidic device according to any of the preceding claims, wherein the microfluidic network contains biological or biomimetic material including one or more of:
a. gel, extracellular matrix or scaffold provided for example in the first sub-volume;
b. epithelial or endothelial cells lining the microfluidic channel, for example forming a tube or blood vessel in the second sub-volume;
c. epithelial or endothelial cells situated inside a gel, extracellular matrix or scaffold, preferably forming lumened structures, more preferably forming a vascular bed;
d. stromal cells in or on a gel, extracellular matrix or scaffold;
e. muscle cells in or on a gel, extracellular matrix or scaffold;
f. one or more other cell types selected from pluripotent cells and central nervous, peripheral nervous, lymphoreticular, immune, urinary, respiratory, reproductive (male and female), gastrointestinal, endocrine, skin, musculoskeletal, cardiovascular, and mammary cell types.

16. A method to assess mechanical strain induced by cells, comprising:
introducing one or more types of cells or cell aggregates into the microfluidic network of a microfluidic device according to any one of claims 1 to 15;
optionally culturing the one or more types of cells or cell aggregates; and
monitoring deflection of the diaphragm using one or more electrodes, sensors, probes, or reference markers for monitoring diaphragm movement, disposed on or operatively connected to the diaphragm.

17. A method of subjecting one or more types of cells or cell aggregates to mechanical strain, comprising:
introducing one or more types of cells or cell aggregates into the microfluidic network of a microfluidic device according to any one of claims 1 to 15;
optionally culturing the one or more types of cells or cell aggregates; and
subjecting the one or more types of cells or cell aggregates to mechanical strain by applying a positive pressure or a negative pressure to the diaphragm.

18. The method of claim 17, comprising applying an alternating positive pressure and negative pressure.

19. The method of any one of claims 17 and 18, wherein mechanical strain is varied through time in a single, cyclical or repeating pattern.

20. The method according to any one of claims 17 to 19, wherein the device comprises a plurality of diaphragms in contact with the microfluidic channel and wherein the plurality of diaphragms are configured such that multiple actuations of one or more of the plurality of diaphragms in a predetermined pattern causes a net fluid movement through the microfluidic network over the course of multiple actuation cycles.

21. The method of any one of claims 16 to 20, comprising:
introducing into the microfluidic network a volume of a gel or gel precursor;
allowing the volume of gel or gel-precursor to cure or gelate to form a cured gel;
loading the microfluidic network with a fluid; and
culturing the one or more types of cells or cell aggregates.

22. The method of any one of claims 17 to 21, wherein the method comprises:
introducing the volume of gel or gel-precursor into the first sub-volume and allowing the volume of gel or gel-precursor to be confined by the capillary pressure barrier.

23. The method of any one of claims 16 to 22, further comprising:
introducing one or more types of cells into the microfluidic channel, preferably including at least one type of epithelial or endothelial cells.

24. The method of any one of claims 21 to 23, further comprising:
introducing one or more types of cells, preferably including at least one type of epithelial cells, to the third sub-volume via the inlet aperture; and allowing the one or more types of cells to form a (mono-)layer or cell aggregate.

25. The method of claim 24, wherein the third sub-volume is fluidically connected to the inlet aperture and is optionally defined at least in part by a surface of the gel.

26. The method of any one of claims 16 to 25, further comprising culture of any one or a combination of:
a. epithelial or endothelial cells lining the microfluidic channels, potentially forming a tube or blood vessel;
b. epithelial or endothelial cells situated inside, on or against a gel, extracellular matrix or scaffold, preferably forming lumened structures, more preferably forming a vascular bed;
c. stromal cells in, on or against a gel, extracellular matrix or scaffold;
d. muscle cells in, on or against a gel, extracellular matrix or scaffold;
e. one or more other cell types selected from pluripotent cells and central nervous, peripheral nervous, immune, urinary, respiratory, reproductive (male and female), gastrointestinal, endocrine, skin, musculoskeletal, cardiovascular, and mammary cell types.
